SEQUENCE LISTING (3 sequences)

SEQ ID NO: 1 - protein, 390 aa, Stenotrophomonas maltophilia
MSNATSQDRALALATLAIHGGQSPDPSTGAVMPPIYATSTYAQSSPGEHQGFEYSRTHNPTRFAYERCVASLEGGTRGFAFASGMAASSTVIELLDAGSHVVAMDDIYGGSFRLFERVRRRTAGLDFSFVDLTDLAAFEASITPKTKMVWIETPTNPMLKIVDIAAVAAIAKRHGLIVVVDNTFASPMLQRPLELGADLVLHSATKYLNGHSDMVGGMVVVGDNAELAEQMAFLQNSVGGVQGPFDSFLALRGLKTLPLRMKAHCANALALAQWLEKHPAVEKVIYPGLASHPQHELAGKQMAGYGGIVSIVLKGGFDAAKRFCEKTELFTLAESLGGVESLVNHPAVMTHASIPVARREQLGISDALVRLSVGVEDLGDLQVDLGEALK

SEQ ID NO: 2 - DNA, 1173 bp, Stenotrophomonas maltophilia
atgtccaacgctacttcccaggatcgtgccctggccctggctaccctggccatccacggtggccagtcgccggaccccagtaccggtgcggtgatgccgccgatctacgctacctcgacctatgcgcagtccagcccgggtgagcaccagggcttcgagtattcgcgtacccacaacccgacgcgcttcgcctacgagcgctgtgtggcctcgctggaaggcggtacccgtggcttcgccttcgcttcgggcatggccgccagctcgaccgtgatcgagctgctggacgccggcagccatgtggtggcgatggatgacatctatggcggcagcttccgcctgttcgagcgtgtgcgtcgccgtaccgccgggctggacttcagcttcgtcgatctgaccgatctggcggcgtttgaagcctccatcacgccgaagacgaagatggtctggatcgagaccccgaccaacccgatgctgaagatcgtcgacatcgctgcggtcgccgccatcgccaagcgccatggcctgatcgtagtggtcgacaacaccttcgcctcgccgatgctgcagcgtccgctggagctgggcgcggacctggtcctgcattcggccaccaagtacctcaatggccactcggacatggtcggcggcatggtcgtggtcggtgacaacgccgaactggccgagcagatggccttcctgcagaactcggtgggtggcgtgcagggcccgttcgacagcttcctggccctgcgtggcctgaagaccctgccgctgcgcatgaaggcgcattgcgccaacgcgctggccctggcccagtggctggaaaagcatccggcggtggagaaggtgatctacccgggcctggcctcgcacccgcaacatgagctggcgggcaagcagatggctggctacggcggcatcgtttcgatcgtgctcaagggcggtttcgatgcggccaagcgcttctgcgagaagaccgaactgttcacccttgccgaatcgctgggcggcgtggaaagcctggtcaaccacccggcggtgatgacgcatgcgtcgattccggtggcgcgtcgtgagcagctgggcatcagcgatgcgctggtgcggctgagtgtgggtgttgaggatctgggggatctgcaggtggatctgggagaggctttgaagtga

SEQ ID NO: 3 - DNA, 1170 bp, artificial sequence
  codon optimized SmCSE gene
atgtcaaatgcaacgagtcaagatcgcgcactggctctggcaacgctggctattcacggtggtcaatccccggacccgagcacgggcgccgtgatgccgccaatttatgcaaccagcacgtacgctcagagctctccgggcgaacatcaaggttttgaatattctcgcacccacaacccgacgcgcttcgcctacgaacgttgcgtggcatcactggaaggcggtacccgtggctttgcattcgcttcgggtatggcggccagttccacggttattgaactgctggatgctggcagccatgtggttgcgatggatgacatctatggcggttcttttcgcctgttcgaacgtgtccgtcgccgtaccgcgggtctggacttttcattcgtggatctgacggacctggcagcttttgaagcgtcgattaccccgaaaacgaaaatggtttggatcgaaaccccgacgaacccgatgctgaaaattgttgatatcgcggccgtcgcagctattgcaaaacgccacggcctgatcgtcgtggttgataataccttcgcgagtccgatgctgcagcgtccgctggaactgggtgccgacctggttctgcatagcgcaaccaaatacctgaacggccactctgatatggtcggcggtatggtcgtggttggtgacaacgcggaactggccgaacagatggcgtttctgcaaaatagtgtcggcggtgtgcagggcccgtttgattccttcctggcgctgcgcggtctgaaaaccctgccgctgcgtatgaaagcccattgcgcgaatgccctggcactggctcaatggctggaaaaacacccggctgttgaaaaagtcatctatccgggcctggcaagccatccgcagcacgaactggcgggcaaacaaatggccggttacggcggtattgtgagcatcgttctgaaaggcggttttgatgcggccaaacgcttctgtgaaaaaaccgaactgtttacgctggccgaatcactgggcggtgtcgaatcgctggtgaatcatccggcagtgatgacccacgctagtattccggttgcgcgccgtgaacagctgggtatctccgacgcgctggtgcgtctgtcggttggcgtggaagacctgggtgacctgcaagttgacctgggtgaagcactgaaa

(19) United States
(12) Patent Application Publication
(10) Pub. No.: US 2017/0335309 A1
(43) Pub. Date: Nov. 23, 2017

(54) ISOLATED ENZYMATIC MANUFACTURE OF SEMICONDUCTOR NANOPARTICLES

(71) Applicants: Bryan Berger, (US); Steven McIntosh, (US)

(72) Inventors: Bryan Berger, Allentown, PA (US); Steven McIntosh, Allentown, PA (US)

(73) Assignee: Lehigh University, Bethlehem, PA (US)

(21) Appl. No.: 15/159,109

(22) Filed: May 19, 2016

(51) Int. Cl. C12N 9/88; C12N 15/74; B82Y 20/00; B82Y 40/00; B82Y 5/00

(52) U.S. Cl. CPC C12N 9/88; C12N 15/74; Y10S 977/824; Y10S 977/774; B82Y 5/00; B82Y 40/00; B82Y 20/00; Y10S 977/95; C12Y 404/01001

(57) ABSTRACT

Novel semiconductor nanoparticles and methods of biosynthesizing the same are provided by biosynthetic processes using cell-free supernatants and isolated enzymes.

### STATEMENT REGARDING GOVERNMENT INTERESTS

This work was supported in part by the following United States Government grants: National Science Foundation under the EFRI-PSBR program, Grant No. 1332349. The Government has or may have certain rights in this invention.

### REFERENCE TO SEQUENCE LISTING SUBMITTED ELECTRONICALLY

The Sequence Listing associated with the application is provided in text format in lieu of a paper copy, and is hereby incorporated by reference into the specification. The name of the text file containing the Sequence Listing is SequenceListing.txt. The text file is 7 kilobytes, was created on May 19, 2016 and is being submitted electronically via EFS-Web.

### FIELD OF THE INVENTION

This invention relates generally to semiconductor nanoparticle biomanufacture.

### BACKGROUND OF THE INVENTION

Without limiting the scope of the invention, its background is described in connection with manufacture and uses of semiconductor nanoparticles including quantum dots (QDs). QD have numerous potential applications in a number of technologies, including display technologies, in vivo or in vitro biomedical imaging/detection, and quantum-dot solar cells. Most current methods to produce QDs utilize elevated temperature, organic solvent based processes. For example, an existing method of manufacturing cadmium sulfite (CdS) QD utilizes bis(trimethylsilyl)sulfide [(TMS)₂S] reacting with dimethylcadmium [Me₂Cd] at 300° C. in an anhydrous environment. Me₂Cd is expensive, toxic, and pyrophoric. An alternative method utilizes trioctylphosphine oxide [TOPO], CdO, and hexylphosphonic acid or tetradecylphosphonic acid at 300° C., again in anhydrous environment. The processes are not particularly efficient and the reported figure of merit, quantum yields (QYs), of the resulting CdS quantum dots vary in the range 3-12%. In addition to environmental issues related to scale-up of these solvent based reactions, the reactants themselves must be synthesized. As such there has been increasing interest in developing aqueous based synthesis methods, commonly based on reactions of cadmium sulfate [CdSO_{4]} with, for example, sodium thiosulphate [Na₂S₂O_{3]} or sodium sulfide [Na₂S].

Biosynthesis and assembly of hard materials, including QDs, has been described previously in both prokaryotes and eukaryotes. Both have evolved several resistance mechanisms in response to toxic levels of heavy metals such as cadmium. These responses include the production of cysteine rich peptides to direct growth of insoluble metal precipitates and the formation of glutathione-metal complexes to trap intracellular metals. These observations have inspired the pursuit of a range of biological approaches to semiconductor nanocrystal biosynthesis, including using peptides, glutathione or other cellular components to direct synthesis.

For example, Li et al., produced a single size of CdSe QDs within yeast cells through genetic engineering of intracellular redox conditions, illustrating the potential for cellular engineering to regulate nanocrystal biosynthesis. See Y. Li, et al., ACS Nano 7 (2013) 2240-2248. While this and several other biological approaches have shown that biomineralization of CdS and related semiconductor nanocrystals can occur, these previous methods demonstrate only limited control over the final particle size or resulted in production of particles with a limited size distribution. Since the opto-electronic functionality of QDs is largely due to their size dependent band gap, any relevant synthesis route must allow reproducible control over the nanocrystal size within the quantum confinement size range.

Bai et al. utilized immobilized Rhodobacter sphaeroides to demonstrate a progressive increase in CdS particle size with increasing cell incubation time, but did not provide information on the size distribution of the particles or the evolution of particle size over time. See H. Bai, et al., Nanoscale Res. Lett. 4 (2009) 717-723. Other reports of biosynthesized CdS nanocrystals show broad size distributions for single growth times. For example, Borovaya synthesized CdS particles with sizes ranging from 2 to 8 nm within a single growth batch. See M. Borovaya, et al., Nanoscale Res. Lett. 9 (2014) 686. These previous reports demonstrate a mixture of both intracellular QDs and extracellular QD production. However, no previous report has demonstrated reproducible, extracellular biomanufacturing of CdS QDs.

Provided herein are methods and compositions of size selected semiconductor QD generated by bacterial culture, extracellular biomanufacturing and biomanufacturing with isolated enzymes.

### BRIEF SUMMARY OF THE INVENTION

Provided herein are novel biosynthetic methods of generating quantum dots as well as the quantum dots produce by biosynthetic processes using cell-free supernatants and isolated enzymes. In one embodiment a method of biosynthesizing a nanoparticle quantum dot material is provided including providing a bacterial organism that is tolerant to a selected metal salt, placing the bacterial organism in an aqueous environment comprising the selected metal salt for a time sufficient for the bacterial organism to utilize the metal salt to assemble semiconductor nanoparticles, removing the bacterial organism to provide a cell-free solution, allowing the semiconductor nanoparticles to continue to grow in the cell-free solution until a desired semiconductor quantum dot population is obtained, and harvesting the desired semiconductor quantum dot population.

In certain embodiments, the semiconductor nanoparticles generated biosynthetically have an average particle size of between about 1 nm to about 10 nm. In certain embodiments, the bacterial organism is selected from a class Gammaproteobacteria and an order Xanthomonadales. Use of a bacterial organism from a genus Stenotrophomonas is exemplified. In certain embodiments the Stenotrophomonas is a species selected from: S. acidaminiphila, S. dokdonensis, S. koreensis, S. maltophilia, S. nitritireducens, and S. rhizophila.

The metal salt may be selected from I-VI, II-VI, IV-VI and III-V semiconductor metals. In one example the metal salt comprises cadmium, and the bacterial organism is tolerant to cadmium concentrations above 1 mM.

In one embodiment a method of generating an enzyme that biosynthesizes nanoparticle quantum dot materials is provided including iteratively culturing and selecting a bacterial organism that is tolerant to growth in the presence of a metal salt that is selected from I VI, II-VI, IV-VI and III-V semiconductor metals and generates controlled particle size quantum dots by culture in the presence of the metal salt. A gene encoding a cystathione gamma (γ)-lyase enzyme is isolated and cloned from the tolerant bacterial organism. The gene is expressed from a heterologous bacterial host and the resulting cystathione gamma (γ)-lyase enzyme is isolated. As an example a cystathione gamma (γ)-lyase enzyme is cloned from a bacteria of the class Gammaproteobacteria, order Xanthomonadales and genus Stenotrophomonas.

In another embodiment, a method of biosynthesizing a nanoparticle quantum dot material is provided that includes incubating a cystathione gamma (γ)-lyase in an aqueous solution comprising L-cysteine and cadmium acetate for a sufficient time to generate a desired quantum dot material and isolating the quantum dot material. In one example, the cystathione gamma (γ)-lyase is a recombinantly produced enzyme isolated from a Stenotrophomonas maltophilia bacteria that was selected for tolerance to growth in the presence of cadmium.

Also provided are biosynthetic nanoparticle quantum dot materials that are enzymatically synthesized by an isolated recombinant cystathione gamma (γ)-lyase in an aqueous solution comprising L-cysteine and a metal salt. In certain embodiments the metal salt comprises a metal selected from the group consisting of Cd, Ce, Cu, Fe, Hg, In, Ga and Zn. In an example provided the metal salt comprises a Cd metal.

### BRIEF DESCRIPTION OF THE DRAWINGS

For a more complete understanding of the present invention, including features and advantages, reference is now made to the detailed description of the invention along with the accompanying figures:

FIGS. 1A and B illustrate absorption and emission spectra of the as-grown cultures according to one embodiment of the present invention. FIG. 1A shows the absorption spectra of a series of samples prepared using different growth conditions, showing that SMCD1 cells grown in the presence of both cadmium acetate and L-cysteine in M9 minimal media result in the formation of CdS QDs with a well-defined first excitonic peak. FIG. 1B shows optical properties of three different batches of CdS QDs prepared using the same growth conditions showing good reproducibility. Inset is a photograph of the culture supernatants illuminated under UV light. The emission spectra were recorded using a 350 nm excitation wavelength.

FIGS. 2A-C represent optical properties of an embodiment of as-grown CdS QDs with different growth times. FIG. 2A presented photograph of the culture supernatants from strain SMCD1 collected at various growth times when illuminated under UV light. FIG. 2B shows UV-vis absorption spectra of CdS QDs as a function of growth time. FIG. 2C shows fluorescence emission spectra using a 350 nm excitation wavelength as a function of growth time.

FIGS. 3A-D show electron microscopy characterization of purified CdS QDs after 60 min growth. FIG. 3A is a bright field TEM image of CdS QDs illustrating the typical particle dispersion. The inset is the corresponding selected area electron diffraction (SAED) pattern. FIG. 3B is a representative high resolution TEM image of CdS QDs. FIG. 3C is a high angle annular dark field (HAADF) image of particles exhibiting zinc-blende type structures respectively. FIG. 3D is a high angle annular dark field (HAADF) image of particles exhibiting wurtzite type structures.

FIG. 4A represents energy-dispersive X-ray spectroscopy (XEDS) analysis confirming the co-existence of Cd and S in the QDs.

FIG. 4B represented an X-ray powder diffraction pattern of the precipitated CdS quantum dot powder after 360 min of growth.

FIGS. 5A-D show particle size distributions and quantum yields of CdS QDs as a function of growth time. FIG. 5A shows representative particle size distributions for growth times of 60 minutes. FIG. 5B shows representative particle size distributions for growth times of 180 minutes. FIG. 5C shows representative particle size distributions for growth times of 300 minutes. In each case the mean values were derived from measurements of at least 100 particles). FIG. 5D shows quantum yield values of CdS QDs with growth times ranging from 30 min to 180 min.

FIGS. 6A and B show optical properties of an embodiment of as-grown CdS QDs as a function of growth time in centrifuge supernatant following removal of cells via centrifugation at 30 minutes. FIG. 6A is a UV-vis absorption spectra. FIG. 6B is a fluorescence emission spectra using a 350 nm excitation wavelength as a function of growth time.

FIG. 7A shows the amino acid sequence of the protein associated with the QDs. The identified protein sequence (NCBI accession number WP_012509966) corresponds to a predicted cystathionine gamma-lyase, based on the results of ESI-MS. Specific peptide sequences from ESI-MS used in protein identification are given in bold and underlined for emphasis. FIG. 7B is the genomic sequence of the S. maltophilia CSE (Smal_0489). FIG. 7C is the nucleic acid sequence of S. maltophilia CSE codon optimized for expression in E. coli. FIG. 7D provides an alignment of the genomic sequence and the codon optimized sequence.

FIGS. 8A and B depict the optical properties of CdS versus synthesis time. FIG. 8A depicts absorbance spectra of smCSE (0.1 mg/mL) incubated with 4 mM L-cysteine and 0.5 mM cadmium acetate for various time intervals. FIG. 8B depicts corresponding fluorescence spectra at selected time intervals using an excitation wavelength of 350 nm. The red-shift of the absorbance and fluorescence maxima indicates an increase in the mean size of the CdS nanocrystals with incubation time.

FIG. 9A shows photographs of photoluminescence under UV light of the effects of smCSE (0.1 mg/mL) incubated with 4 mM L-cysteine and 0.5 mM cadmium acetate for various time intervals.

FIG. 9B shows the results of a High Angle Annular Dark Field—Scanning Transmission Electron Microscopy (HAADF-STEM) characterization of smCSE synthesized nanocrystals and depicts the XEDS spectrum confirming the co-existence of Cd and S in the nanocrystals.

FIGS. 10A-C show further results of a High Angle Annular Dark Field—Scanning Transmission Electron Microscopy (HAADF-STEM) characterization of smCSE synthesized nanocrystals. FIG. 10A shows a representative image showing numerous dispersed but overlapping nanocrystal. FIG. 10B shows example images from wurtzite CdS nanocrystals viewed along [101] and [211] projections, respectively. FIG. 10C shows example images from zinc-blende CdS nanocrystals viewed along [110] and [211] projections, respectively.

FIGS. 11A-D show control of nanocrystal size by the co-addition of L-cysteine and glutathione. FIG. 11A shows absorbance maximum versus reaction time for preparations containing 4 mM, 10 mM, and 20 mM of L-cysteine. FIG. 11B shows the corresponding photographs of the solutions in FIG. 11A under UV illumination. FIG. 11C shows the optical absorbance maximum versus reaction time for 4 mM L-cysteine supplemented with 1 mM, 4 mM, and 10 mM of glutathione. FIG. 11D shows the corresponding photographs of the solutions in FIG. 11C under UV illumination. For all syntheses smCSE and cadmium acetate concentrations were kept constant at 0.1 mg/mL and 0.5 mM respectively.

FIGS. 12A-C show that smCSE forms nanocrystals in the absence of L-cysteine or glutathione. FIG. 12A shows UV-Visible absorbance spectra obtained upon the addition of 4 mM Na₂S to a preparation containing 0.5 mM cadmium acetate in the presence of smCSE, 4 mM L-cysteine or 4 mM glutathione. A solution of Na₂S added to 0.5 mM cadmium acetate is shown as a control. FIG. 12B shows corresponding fluorescence (excitation at 360 nm) of solutions in FIG. 12A showing photoluminescence under UV light. FIG. 12C is a schematic of proposed CdS quantum dot synthesis by smCSE. smCSE associates with cadmium acetate and L-cysteine present in solution, smCSE degrades L-cysteine to produce H₂S, H₂S produced by smCSE nucleates CdS nanoparticles and CdS nanoparticles continue to grow upon the continuous generation of H₂S.

### DETAILED DESCRIPTION OF THE INVENTION

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

The present inventors appreciated the need for and developed reproducible, extracellular biomanufacturing of semiconductor quantum dot nanoparticles (QDs) using an isolated enzyme that both mineralizes metal ion-counterion combinations and templates nanocrystal formation. As used herein, the interchangeable terms “nanoparticle” and “nanocrystal” refer to a particle between 1 and 100 nanometers in at least one dimension. As used herein, the term “quantum dot” refers to a nanoparticle between 1 and 50 nanometers in at least one dimension, wherein nanoparticle comprises a fluorescent semiconductor material in which electron propagation is confined in three dimensions.

The quantum dots provided herein are useful to provide imaging and lighting in many technological applications. For example, semiconductor quantum dots have been used as biocompatible probes for in vivo imaging and medical diagnostics, as potential replacements or enhancers to LED lighting, as modifiers or replacements in LED display technology, as active materials in photovoltaic cells (so-called quantum dot solar cells), and as potential catalysts for water splitting (i.e., hydrogen generation) for fuel cell applications, as well as in semiconductors, biomedical diagnostics, imaging, targeting and drug delivery, biosensors, lighting, display technology, solar cells, and photovoltaics, for example.

A major barrier to the utilization of quantum dots in commercial applications is the high cost associated with conventional chemical synthesis due to high temperatures, pressures and toxic solvents, thereby requiring specialized, expensive waste disposal procedures. Furthermore, multi-stage synthesis methods are necessary to ‘cap’ chemically-synthesized QDs in order to enhance water solubility. Therefore, more cost-efficient and environment friendly methods of producing and using soluble quantum dots, as well as less toxic quantum dot compositions, are desirable.

In one embodiment provided herein, a novel alternative aqueous-based, bacteria mediated, biosynthetic procedure is detailed for CdS nanocrystal synthesis at 37° C. As an illustrative but non-limiting embodiment, relatively inexpensive precursor cadmium acetate [Cd(CH₃CO₂)_{2]} was utilized as a Cd source and the amino acid L-cysteine as the sulphur source and capping agent. The resulting nanoparticles were shown to exhibit quantum properties and were thus shown to be quantum dots that show QYs approaching those of the chemical synthesized materials.

A distinguishing feature of the example provided herein is the demonstration of reproducible CdS nanocrystal biosynthesis with control over the mean particle size largely in the 2-4 nm range, providing access to a wide range of quantum confined band gap energies. While the mean size and distribution width increases with increasing growth time, the as-synthesized standard deviation in mean size is demonstrated to be still less than 1 nm.

In one illustrative embodiment, this was achieved by utilizing directed evolution to engineer a bacterial strain that is capable of extracellular production of CdS nanocrystals. The developed and demonstrated extracellular production simplifies purification. Stenotrophomonas maltophilia was specifically chosen because of its intrinsically high resistance to a variety of heavy metals, including cadmium, although the process could be applied to other bacteria exhibiting this property. The nanocrystals produced are confirmed to be crystalline CdS via high resolution scanning transmission electron microscopy, X-ray energy dispersive spectroscopy (XEDS), and X-ray diffraction. Following this, the enzyme responsible for generation of the CDS QD was identified by the present inventors.

As such it was determined that a cystathionine γ-lyase (smCSE) was associated with the extracellular synthesis of CdS quantum dot nanocrystals by Stenotrophomonas maltophilia strain SMCD1. Cystathionine γ-lyases (CSEs) are a class of enzymes that catalyze the production of H₂S, NH₃ and pyruvate from L-cysteine, and the overexpression of which has been shown to precipitate cadmium sulfide. As provided herein, we demonstrate the smCSE from S. maltophilia SMCD1 is capable of reactive H₂S generation, consistent with its function as a cystathionine γ-lyase. In addition, the purified smCSE enzyme, by itself, was shown to be capable of aqueous phase synthesis of CdS nanocrystals directly from cadmium acetate and L-cysteine. The resulting CdS nanocrystals are within the quantum confined size range and display optoelectronic properties analogous to those previously described for cell-based or chemically-synthesized CdS nanocrystals. When the substrate L-cysteine is replaced by the chemical precursor Na₂S, smCSE is capable of directing CdS nanocrystal formation in solution. Removal of smCSE results in bulk CdS formation, indicating a role for smCSE not only in H₂S generation, but also in templating CdS nanocrystals. This demonstrates that a single enzyme is capable of synthesizing metal sulfide nanocrystals directly from aqueous solution, opening up a wide range of strategies for engineering the biomineralization of functional materials.

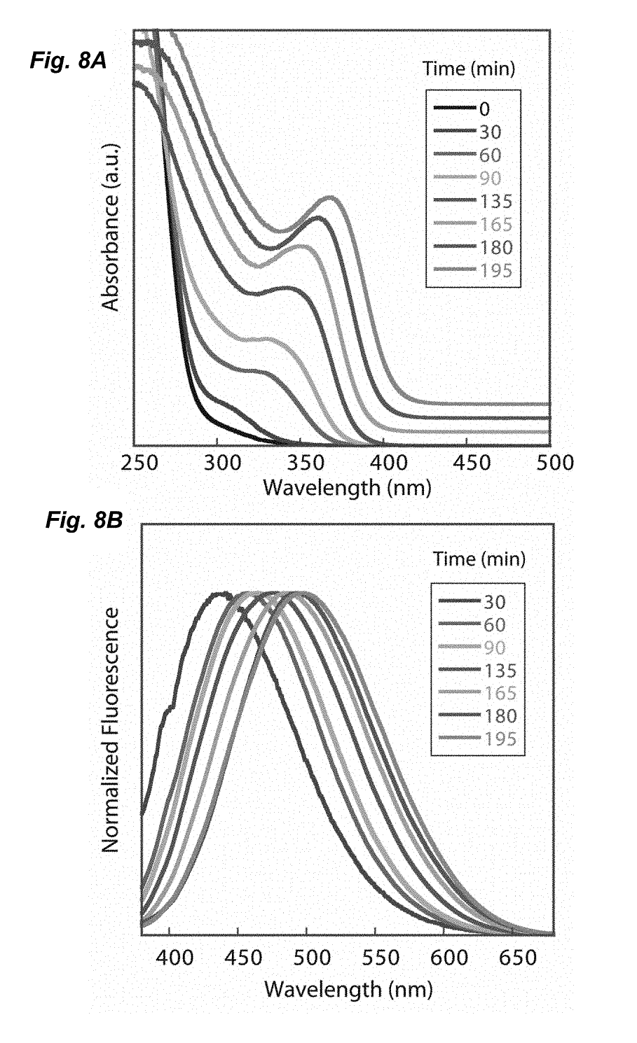

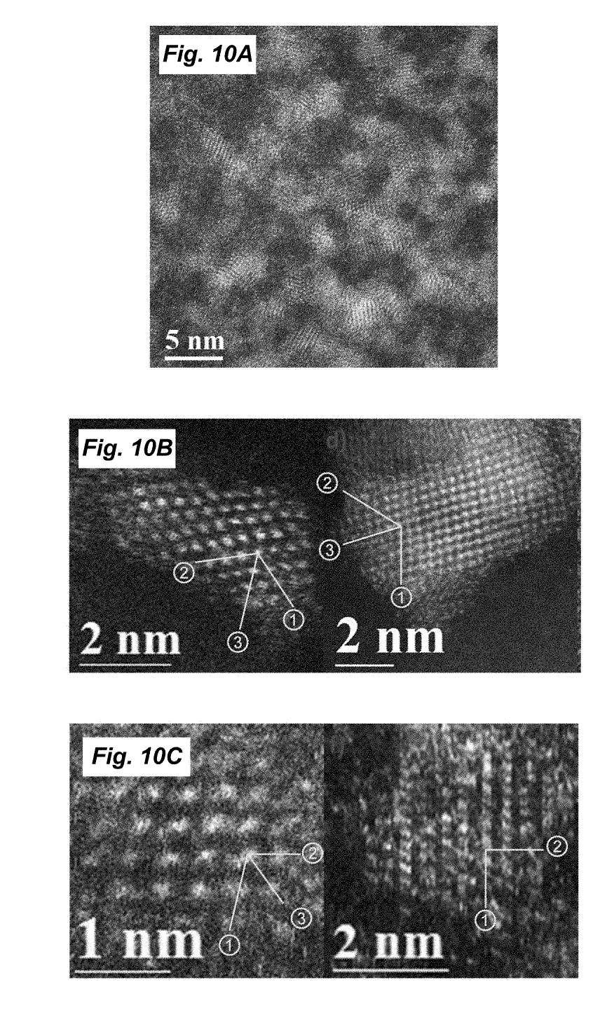

The present disclosure clearly demonstrates the ability of a single enzyme (smCSE) to produce both crystalline CdS (FIGS. 10A-C) and to regulate growth to form nanocrystals within the quantum confined size range (FIGS. 8A and B). Previous studies involving enzymatic or peptide-based biomineralization of metal sulfide nanoparticles have demonstrated either CdS formation from enzymatically generated H₂S without intrinsic size control (Ansary A A, et al. CdS quantum dots: Enzyme mediated in vitro synthesis, characterization and conjugation with plant lectins. J Biomed Nanotechnol 3(4) (2007) 406-413; Wang C L, et al. Aerobic sulfide production and cadmium precipitation by Escherichia coli expressing the Treponema denticola cysteine desulfhydrase gene. Appl Microbiol Biotechnol 56 (2001) 425), or have demonstrated nanocrystal size control by adding specific peptides or proteins and utilize reactive Na₂S as a sulfur source. See Spoerke E D, Voigt J A. Influence of engineered peptides on the formation and properties of cadmium sulfide nanocrystals. Adv Funct Mater 17 (2007) 2031-2037; Bae W, Mehra R K. Properties of glutathione- and phytochelatin-capped CdS bionanocrystallites. J Inorg Biochem 69 (1998) 33-43; and Liu F, et al. Enzyme mediated synthesis of phytochelatin-capped CdS nanocrystals. Appl Phys Lett 97 (2010) 123703.

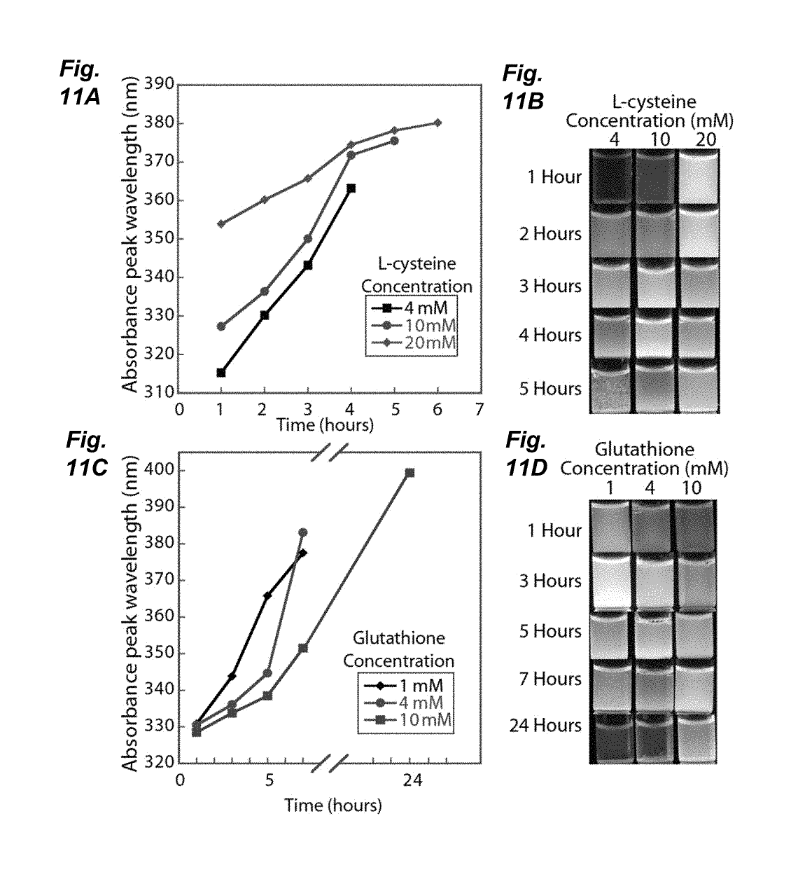

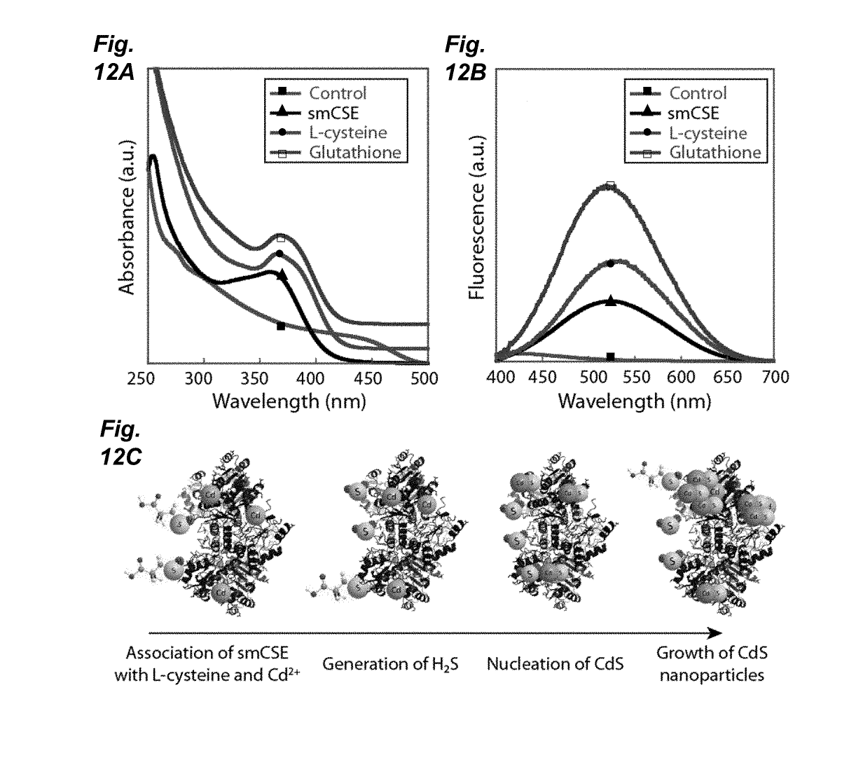

A remarkable property of the smCSE disclosed herein is that the enzyme acts both as the sulfur generating source to mineralize CdS (FIG. 8A), and as structure directing agent to control nanocrystal growth (FIGS. 12A and B). Thus, smCSE efficiently combines mineralization and templating into a single enzyme (smCSE; FIG. 12C) that are engineered separately in other biomineralization strategies. The combination of H₂S generation and controlled growth afforded by smCSE is extraordinary compared to other enzymatic and peptide approaches to nanocrystal synthesis. In the presence of excess capping agents (L-cysteine or glutathione) under conditions where smCSE can generate H₂S, we found that the CdS nanocrystals can span a range of quantum confined sizes and resulting optical properties) and that reducing the amount or quality of capping agent limits the stability of the resulting colloidal solution (FIGS. 11A-D). It is noted that the addition of capping agents L-cysteine or glutathione appear to improve the nanocrystal quality as well through providing capping agents with smaller size and higher affinity for the particle surface. Independent of its role in H₂S generation, smCSE is also capable of templating CdS nanocrystals from solution when using chemical precursors such as Na₂S (FIGS. 12A-B), to exhibit intrinsic structure directing and capping activity similar to naturally-occurring phytochelatins or nanocrystal-binding peptides.

In a previous study using the protein pepsin as a templating agent (Yang L, et al. Biomimetic synthesis of CdS nanocrystals in aqueous solution of pepsin. Mater Lett 59 (2005)2889-2892), the rate of reactive sulfur generation and subsequent nanocrystal synthesis was at least an order of magnitude slower than that of the smCSE disclosed herein (days versus hours). This rate is dictated by the slow, spontaneous hydrolysis of thioacetamide to generate reactive sulfur, in contrast to the smCSE catalyzed H₂S generation demonstrated in the present work.

In one embodiment an engineered cystathionine γ-lyase (smCSE) is provided that is capable of controlled CdS nanocrystal synthesis directly from aqueous solution using L-cysteine and cadmium acetate as reactants. Furthermore, the ability of smCSE to mineralize CdS and template nanocrystal formation provides a single enzyme route for engineered nanocrystal biomineralization.

As exemplified herein CdS quantum dots were produced by isolated smCSE. However, through the process described herein, other microorganisms are isolatable that may be selected for growth in and detoxification of high concentrations of other metals including Hg, Zn and Cu by precipitating the metals as crystallites in which the metal ion is combined with a counterion. Selection may also be applied to detoxify other metal ions such as Ga and In that are useful in semiconductor generation. In addition to sulfur (S), selenium (Se) and tellurium (Te), and combinations thereof, may also be used as the counterion for the metal. These elements may be provided as reduced forms, for example, sulfate, selenate, and tellerate, or in any compound that provides S, Se, and Te to the organisms.

A common feature of the bacteria used in the present methods is that they ingest a metal salt comprising at least one metal that is useful in forming a semiconductor. For example, metals useful in forming semiconductors include, but are not limited to, the group of I-VI, II-VI, IV-VI and III-V semiconductors as listed in the periodic table of the elements and known to those skilled in the art. By way of further non-limiting example, cadmium is useful as such a metal. In other embodiments the metal is selected from the group consisting of Cd, Ce, Cu, Fe, Hg, In, Ga and Zn.

By way of still further non-limiting example, cadmium from cadmium sulfide (group II-VI) and cadmium from cadmium selenide (group II-VI) is compatible with formation of a semiconductor. In any case, the bacteria selected is tolerant to such metal salts, and either is, or quickly becomes, tolerant when exposed to high concentrations of the selected metal and metal salt. As used herein, “tolerant” means a colony of bacteria grow (i.e., cells undergo division to increase the total number of cells in culture over time) in an aqueous solution of the target metal salt (such as cadmium acetate, for example) at a concentration greater than 1 mM. “Moderately tolerant” as used herein means that the bacteria survive and grow (i.e., cells undergo division to increase the total number of cells in culture over time) at a concentration of the metal salt of greater than 1 mM and up to 5 mM. The term “highly tolerant” or “hyper-tolerant” as used herein means the bacteria survive and grow (i.e., cells undergo division to increase the total number of cells in culture over time) at a concentration of the metal salt of greater than 5 mM.

As exemplified herein, the facile synthesis and purification of large quantities of semiconductor nanoparticles from aqueous solutions is disclosed through direct fermentation using a bacteria that is one of the phylum Proteobacteria. Preferably, the bacteria is also one of the class of Gammaproteobacteria. More preferably, the bacteria is also one of the order of Xanthomonadales. More preferably, the bacteria is also one of the family Xanthomonadaceae. More preferably, the bacteria is also one of the genus: Stenotrophomonas. More preferably, the bacteria is also one of the species S. acidaminiphila, S. dokdonensis, S. koreensis, S. maltophilia, S. nitritireducens, and S. rhizophila.

By way of further example, families of bacteria that are compatible with the present invention are those of the families: Frateuria, Luteimonas, Lysobacter, Nevskia, Pseudoxanthomonas, Rhodanobacter, and Xylella.

By way of further example, bacteria that are compatible further include: Order: Pseudomonadales, Family: Pseudomonadaceae, Genus: Pseudomonas, and Species: P. aeruginosa group, such as: P. aeruginosa, P. alcaligenes, P. anguilliseptica, P. argentinensis, P. borbori, P. citronellolis, P. flavescens, P. mendocina, P. nitroreducens, P. oleovorans, P. pseudoalcaligenes, P. resinovorans, P. straminea, P. chlororaphis group, P. agarici, P. asplenii, P. aurantiaca, P. aureofaciens, P. chlororaphis, P. corrugate, P. fragi, P. lundensis, P. taetrolens, P. fluorescen, [group] P. Antarctica, P. azotoformans, ‘P. blatchfordae’, P. brassicacearum, P. brenneri, P. cedrina, P. corrugate, P. luorescens, P. gessardii, P. libanensis, P. mandelii, P. marginalis, P. mediterranea, P. meridian, P. migulae, P. mucidolens, P. orientalis, P. panacis, P. proteolytica, P. rhodesiae, P. synxantha, P. thivervalensis, P. tolaasii, P. veronii, P. pertucinogena group, P. denitrificans, P. pertucinogena, P. putida group, P. cremoricolorata, P. fulva, P. monteilii, P. mosselii, P. oryzihabitans, P. parafulva, P. plecoglossicida, P. putida, P. stutzeri group, P. balearica, P. luteola, P. stutzeri, P. syringae group, P. amygdale, P. avellanae, P. caricapapayae, P. cichorii, P. coronafaciens, P. ficuserectae, P. helianthi, P. meliae, P. savastanoi, P. syringae, ‘P. tomato’, P. viridiflava, incertae sedis, P. abietaniphila, P. acidophila, P. agarici, P. alcaliphila, P. alkanolytica, P. amyloderamosa, P. asplenii, P. azotifigens, P. cannabina, P. coenobios, P. congelans, P. costantinii, P. cruciviae, P. delhiensis, P. excibis, P. extremorientalis, P. frederiksbergensis, P. fuscovaginae, P. gelidicola, P. grimontii, P. indica, P. jessenii, P. jinjuensis, P. kilonensis, P. knackmussii, P. koreensis, P. lini, P. lutea, P. moraviensis, P. otitidis, P. pachastrellae, P. palleroniana, P. papaveris, P. pelf, P. perolens, P. poae, P. pohangensis, P. protegens, P. psychrophila, P. psychrotolerans, P. rathonis, P. reptilivora, P. resiniphila, P. rhizosphaerae, P. rubescens, P. salomonii, P. segitis, P. septic, P. simiae, P. suis, P. thermotolerans, P. toyotomiensis, P. tremae, P. trivialis, P. turbinellae, P. tuticorinensis, P. umsongensis, P. vancouverensis, P. vranovensis, P. xanthomarina.

In one embodiment the organism is a Stenotrophomonas maltophilia. S. maltophilia has been shown by the inventors to generate novel nanoparticles of relevant materials that are believed to be unable to be synthesized by conventional chemical (non-biological) methods, and having unique properties such as high water solubility that are believed to result from the solubilizing/capping agent used by the selected bacteria.

In one embodiment, an isolated gene encoding a cystathionine γ-lyase (smCSE) is produced in a host that differs from (is heterologous to) the bacterial type from which the gene was isolated. Host cells may be any that may be transformed to allow the expression and secretion of a cystathionine γ-lyase (smCSE). The host cells may be bacterial, mammalian cells, yeast, or filamentous fungi. Various suitable bacteria include Escherichia and Bacillus. Yeasts belonging to the genera Saccharomyces, Kiuyveromyces, Hansenula, or Pichia would be appropriate host cells as would various species of filamentous fungi including the following genera: Achlya, Aspergillus, Cephalosporium, Cochliobolus, Endothia, Mucor, Neurospora, Penicillium, Podospora, Pyricularia And Trichoderma.

In certain embodiments the isolated gene is codon optimization for expression in the heterologous expression system including but not limited to E. coli and yeast expression systems. In an embodiment exemplified herein a gene encoding a Stenotrophomonas maltophilia cystathionine γ-lyase (smCSE) was isolated and codon optimized for expression in E. coli. The exemplified codon optimized gene is 80% homologous with the isolated gene. A codon optimization that includes fewer or more optimized codons would be within the scope of the invention. In other embodiments the gene is codon optimized for expression in a yeast expression system and the cystathionine γ-lyase is produced in bulk fermentation in yeast. The enzyme may be engineered for intracellular or extracellular expression.

Once expressed, the recombinant cystathionine γ-lyase is purified by protein purification techniques well known to those of skill in the art. These techniques may involve purification from clarified supernatants of the host cells or the homogenization and crude fractionation of the host cells. The recombinant cystathionine γ-lyase may be further purified using chromatographic and/or electrophoretic techniques to achieve partial or complete purification. As used herein, a purified protein is intended to refer to a composition wherein the protein or peptide is purified to any degree relative to its naturally-obtainable state and which composition substantially retains its expressed biological activity. Where the term “substantially purified” is used, this refers to a composition in which the protein or peptide forms a major component of the composition, such as constituting about 50%, about 60%, about 70%, about 80%, about 90%, about 95%, or more of the proteins in the composition.

Although the exemplified protein was purified by metal affinity chromatography by virtue of an expressed His tag, various techniques suitable for use in protein purification are well known to those of skill in the art including, for example, precipitation with ammonium sulphate, PEG, antibodies and the like, followed by centrifugation; chromatography steps such as ion exchange, gel filtration, reverse phase, hydroxyapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

The following examples are include for the sake of completeness of disclosure and to illustrate the methods of making the compositions and composites of the present invention as well as to present certain characteristics of the compositions. In no way are these examples intended to limit the scope or teaching of this disclosure.

### EXAMPLE 1

### Isolation and Growth of Microorganisms

In one embodiment, a strain of Stenotrophomonas maltophilia was utilized that was isolated from soil collected from the mountaintop campus of Lehigh University in Pennsylvania using conventional methods. Strain identification was confirmed using 16S rRNA sequencing (SeqWright). Standard microbiology techniques were used for the growth and cultivation of Stenotrophomonas maltophilia using Luria-Bertani (LB) broth and M9 minimal media. Selection of cadmium resistant strains was performed iteratively in three steps by increasing the concentration of cadmium acetate to in excess of 1 millimolar (mmol or mM): (1) cultures were grown for 8-12 h at 37° C. in an orbital shaker in LB broth containing increasing concentrations of cadmium acetate (Cd(Ac)₂ at 0.1-5 mM; (2) serial dilutions of cultures were plated onto LB-agar plates containing equivalent concentrations of cadmium acetate; and (3) individual colonies were isolated from plates. Cell growth rate in culture was measured by monitoring the change in optical density at 600 nm (OD600). Colonies tolerant to cadmium acetate at concentrations in excess of 1 mM were selected from cadmium-containing plates. Using this selection procedure, a specific strain (referred to herein as SMCD1) was isolated that exhibits continuous formation of luminescent particles.

In a typical experiment, the selected SMCD1 were subcultured into LB broth (100 mL) and grown for 12 h at 37° C. with shaking. Cells were isolated by centrifugation and resuspended in M9 minimal media (100 mL, OD600=0.5). Then, cadmium acetate (1 mM) and L-cysteine (8 mM) were added and the mixture was placed in a 37° C. incubator with shaking. Sample aliquots were collected every 30 min, and CdS QDs separated from intact cells by centrifugation (5000 rpm), dialysis (Snakeskin 3500 MWCO; Thermo Pierce) with ultrapure, deionized water as the buffer and gravity-feed size exclusion chromatography (PD-10; Amersham).

In order to investigate the mechanism of particle growth, the same growth procedure was followed for 30 minutes growth time. At this point, the cell solution was centrifuged at 8000 rpm for ten minutes, reducing the optical density at 600 nm to 2% of the original value. The centrifuge supernatant was then returned to the incubator at 37° C. and sample aliquots were collected every 30 min.

In one example, a synthesized batch of QDs was separated from cells via centrifugation after 90 minutes of growth time. The supernatant was dialyzed against DI water for 24 hours prior to lyophilisation. The dry sample was analysed via electrospray ionization mass spectrometry.

The luminescence of purified QD suspensions was analyzed using a UV Bio-Rad Gel Doc 2000 system. Absorption spectra of QD suspensions were collected using an Ultrospec 3300 Pro (Amersham Biosciences). Fluorescence excitation and emission spectra for QD suspensions were collected using a Cary Eclipse Fluorescence Spectrophotometer (Varian). Room temperature photoluminescence quantum yields (PL QYs) were calculated using coumarin 1 in ethanol as a standard with a PL QY of 0.73. Powder XRD measurements (Rigaku Miniflex II) were performed at room temperature by using Cu Kα (1.5418 Å) radiation.

For scanning transmission electron microscopy (STEM), selected area electron diffraction (SAED) and X-ray energy dispersive spectroscopy (XEDS) analysis, purified samples were prepared by drop casting the aqueous QD suspension onto a holey carbon-coated copper grid and allowing the liquid component to fully evaporate. The specimen was then analyzed in either (i) a 200 kV aberration corrected JEOL ARM 200CF analytical electron microscope equipped with a Centurio XEDS system or (ii) a 200 kV JEOL 2000FX conventional TEM equipped with an Oxford Instruments XEDS system.

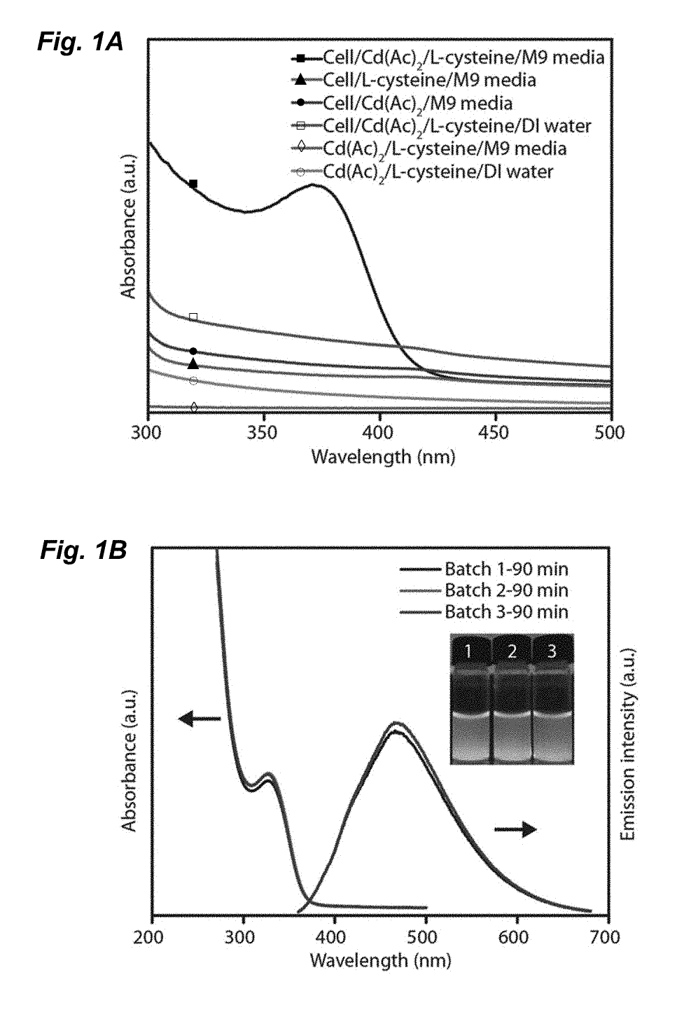

In one illustrative embodiment, Stenotrophomonas maltophilia was isolated from soil and iteratively selected in culture for variants that were tolerant to cadmium acetate at concentrations in excess of 1 mmol. Cadmium-tolerant colonies were selected from cadmium-containing plates, and cultures grown in M9 minimal media containing 8 mmol L-cysteine. From the observed photoluminescence and absorption spectra (FIG. 1A) with 6 h growth, SMCD1 cells grown in the presence of both cadmium acetate and L-cysteine in M9 minimal media resulted in fluorescence consistent with the formation of CdS QDs with a well-defined first excitonic peak. The procedure is reproducible, with essentially identical QD optical properties observed from independent cultures using optimized growth media and the same growth time (FIG. 1B).

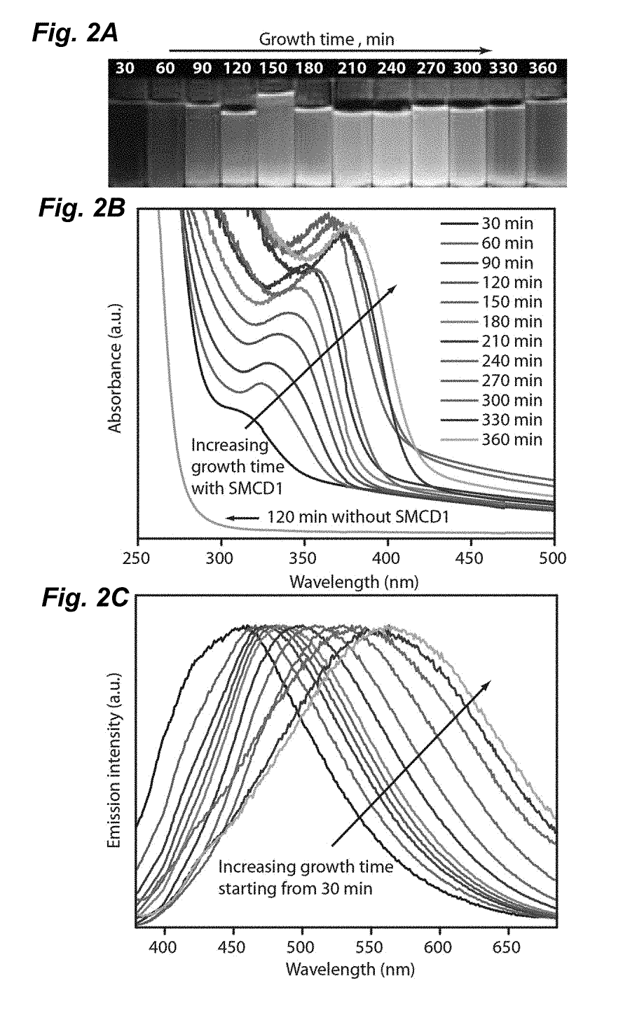

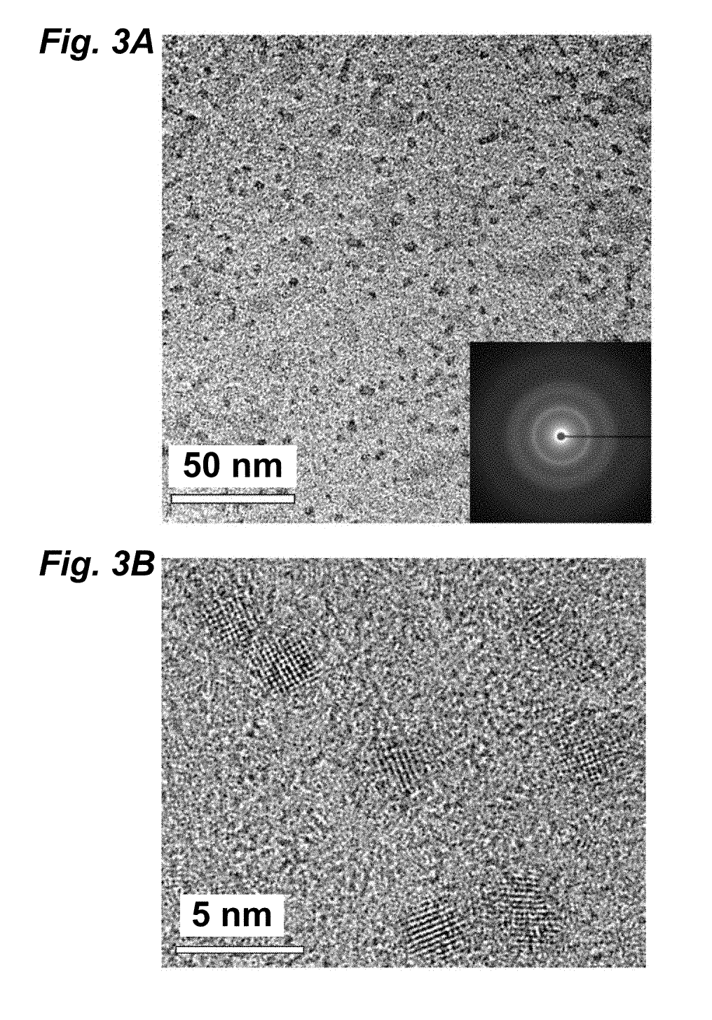

Using SMCD1 cells grown in M9 media including 1 mM Cd(Ac)₂ and 8 mM L-cysteine, it was found that photoluminescence was retained in the culture supernatants after removal of the cells by centrifugation, indicating that the water-soluble fluorescent particles are produced extracellularly (FIG. 2A). It was also found that both absorption and fluorescence peaks shift systematically with increasing growth time in culture. Absorption spectra for samples with various growth times demonstrate well-defined first excitonic peaks (FIG. 2B) with maxima that shift to higher wavelengths with increasing growth time. The normalized fluorescence emission spectra (FIG. 2C) also show a shift to higher wavelength with increasing growth time. For the absorption spectra, the peak wavelength increases from 312 nm to 378 nm, while the corresponding emission spectra peak wavelength moves from 460 nm to 562 nm as the growth time is increased from 30 min up to 360 min.

The formation of water soluble CdS quantum dots was demonstrated in that: 1) both cadmium acetate and L-cysteine are required for fluorescence to be observed in culture, indicating that the fluorescent species is CdS; 2) the requirement for bacterial growth indicates biomineralization of CdS; 3) the measured absorption wavelength maxima are consistent with a blue-shift from the bulk CdS band edge value (˜515 nm) as expected for quantum confined CdS nanoparticles (FIG. 2B) and the emission wavelengths are consistent with broad trap emission from such particles; 4) the observed red-shift in both adsorption and emission wavelengths are consistent with increasing mean size of quantum confined particles with increasing time in culture (FIG. 2C). The relatively large Stokes shift is typical of L-cysteine capped CdS QDs in aqueous solution.

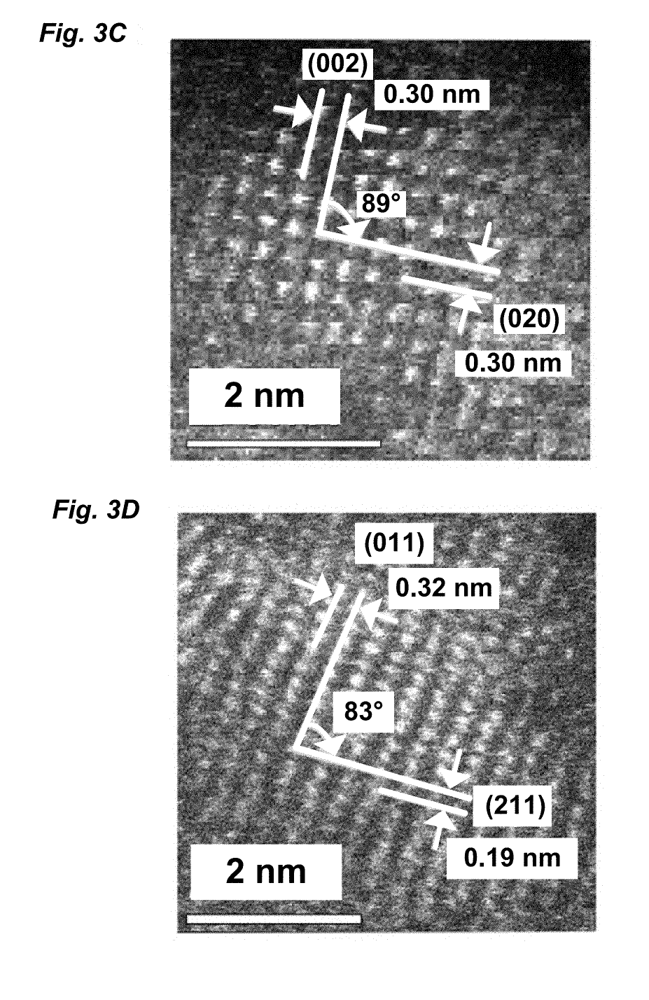

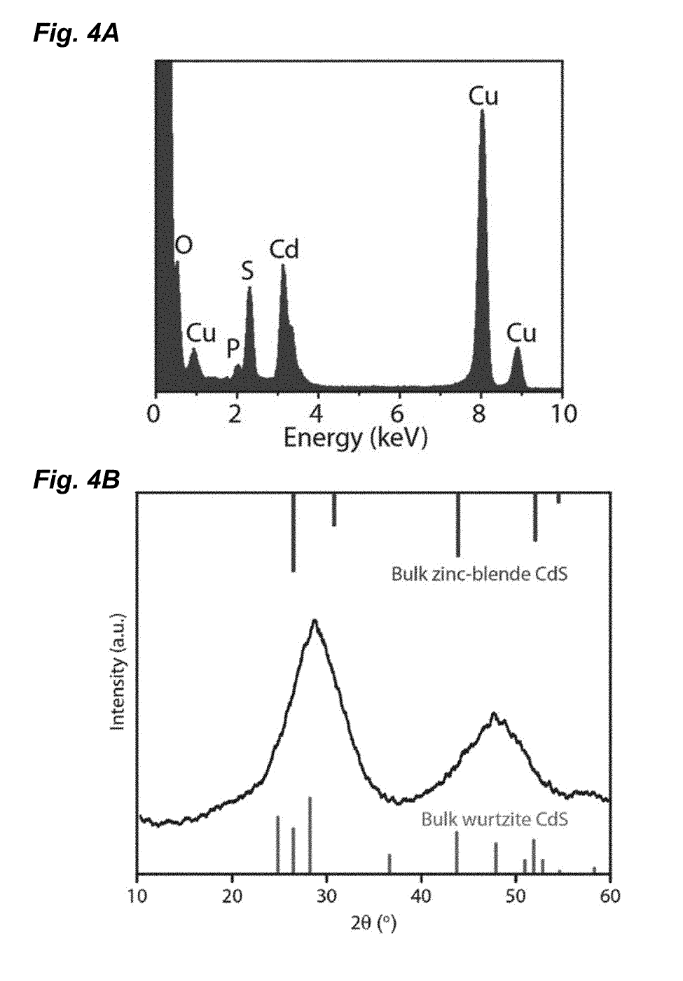

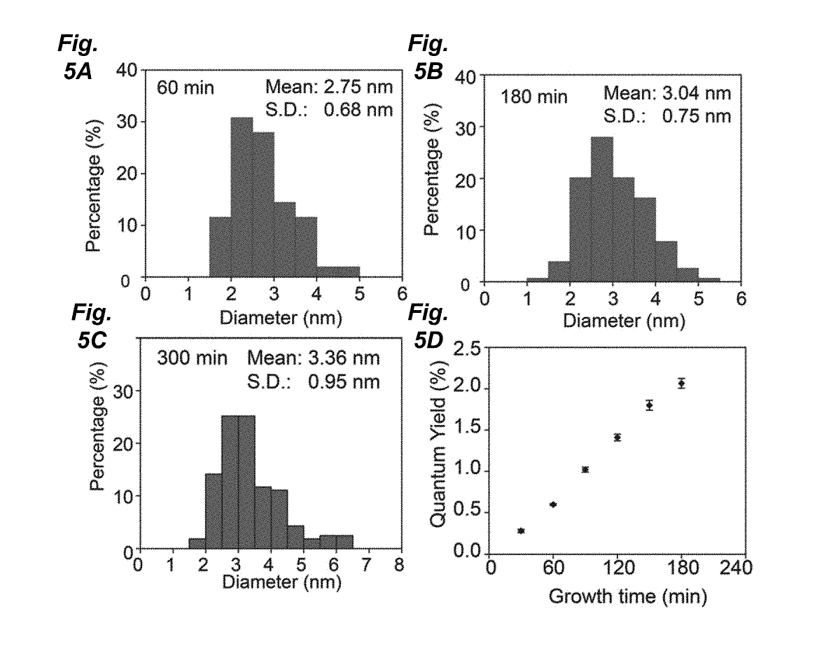

Additional evidence showing biosynthesis of CdS QDs from strain SMCD1 was obtained by high resolution transmission electron microscopy (HRTEM) imaging of the resultant CdS QDs. Specifically, to evaluate the crystal structure and particle size distribution of the CdS materials, nanocrystals produced at a representative growth time of 60 min were purified and characterized by HRTEM (FIGS. 3A and 3B). The highly dispersed QDs are clearly observable (FIG. 3A) by imaging. From electron diffraction (FIG. 3A, inset), two broad but distinct rings corresponding to interplanar spacings of 0.33 nm and 0.21 nm are observed, which are consistent with expected lattice spacings of CdS. However, selected area electron diffraction (SAED) cannot unequivocally distinguish between the possible CdS polymorphs (i.e. the zinc-blende and wurtzite type structures), as they both have lattice spacings similar to those measured within the limits of experimental error. X-ray powder diffraction patterns (FIG. 3C) were also collected and are consistent with CdS formation; however peak broadening due to the nanoscopic nature of the particles results in just two major broad peaks from which again the exact polymorphic structures present cannot be distinguished. HRTEM and STEM-HAADF (high angle annular dark field) images were acquired to obtain more localized crystallographic information from the biosynthesized QDs. The HRTEM (FIG. 3B) and STEM-HAADF images (FIGS. 3C and D) exhibit lattice fringes within individual particles, whose spacings and intersection angles are consistent in some particles with the sphalerite form of CdS whereas in others they match the wurtzite form. The measured d-spacing value of 0.30 nm (FIG. 3C) for the two planes indicated match those of the (002) and (020) planes in zinc-blende type CdS viewed along the [100] projection; the measured interplanar angle is 89°, which matches the expected 90° angle between these two planes. In FIG. 3D the measured d-spacing values of 0.19 nm and 0.32 nm corresponds to those of the (21̅1) plane and (011) plane of wurtzite-type CdS, respectively; the measured interplanar angle between these two planes is 83°, which is also consistent with the expected value of 82.0° calculated from the cross product of (21̅1) and (011) when viewed along [1̅1̅1]. Therefore, it was determined that both zinc-blende and wurtzite type structures co-exist for CdS QDs produced by strain SMCD1. X-ray energy dispersive spectroscopy (XEDS) analysis confirmed that the particles are primarily comprised of cadmium and sulfur (FIG. 4A); some traces of phosphorus and oxygen are also present, most likely due to residual phosphate from the M9 minimal growth media; the copper peaks are artefacts of the TEM support grid.

FIG. 4B shows the X-ray powder diffraction pattern of the precipitated CdS quantum dot powder after 360 min of growth. The stick patterns show the expected standard peak positions of the bulk wurtzite (bottom, PDF card no. 00-006-0314) and zinc-blende type CdS polymorphs (top, PDF card no. 00-010-0454).

Particle size distributions (FIGS. 5A-C) after 60, 180 and 300 min growth times were determined from analysis of such phase contrast images. The mean particle size increases from 2.75 to 3.04 to 3.36 nm after 60, 180 and 300 min, respectively.

The breadth of the distribution also increases with increasing growth time with the standard deviation increasing from 0.68 to 0.95 nm between 60 and 300 min growth. The observed relationship between size and adsorption peak wavelength are in-line with other reports for L-cysteine capped CdS QDs.

The quantum yields (QY) of the purified CdS QDs which had growth times ranging from 30 min to 180 min were found to exhibit an approximately linear increase from 0.30% to 2.08% (FIG. 5D). Determination of the QY at longer growth times was inaccurate due to aggregation of the larger particles. The range of measured QY is once again consistent with other reports for L-cysteine capped QDs in aqueous solution and most importantly are about three orders of magnitude greater than the QY (0.007%) reported for previous biosynthetic CdS QDs.

The relatively low QY as compared to more standard inorganic preparation methods may be due to quenching by the L-cysteine or the dimer cystine. Taking into account the measured systematic variation in particle size distribution with growth time, it can be concluded that the QY monotonically increases with increasing mean nanocrystal size in the 30-180 min growth time range. Similar size-dependent trends for the photoluminescence QY of semiconductor nanocrystals have been noted previously. As the particle size decreases, the surface-to-volume ratio increases, which results in a higher proportion of surface defects. Therefore, it is likely that nonradiative relaxation at surface traps become more important with decreasing particle size.

CdS QDs generated from SMCD1 show an extracellular growth period following cluster nucleation. In yeast, the removal of intracellular cadmium occurs through a glutathione-dependent mechanism, in which up-regulation of glutathione monomer, oligomer and cysteine-rich binding peptide biosynthesis occurs to neutralize cadmium through the formation of metal-thiol complexes. Decomposition of bound thiol ligands, in this case the thiol group on L-cysteine, is thought to provide a source of sulfur for the CdS nanocrystal nucleation and growth. L-Cysteine is one of three amino acids required for glutathione biosynthesis, and the rate of L-cysteine biosynthesis is typically limiting in terms of the overall cellular glutathione biosynthesis rate.

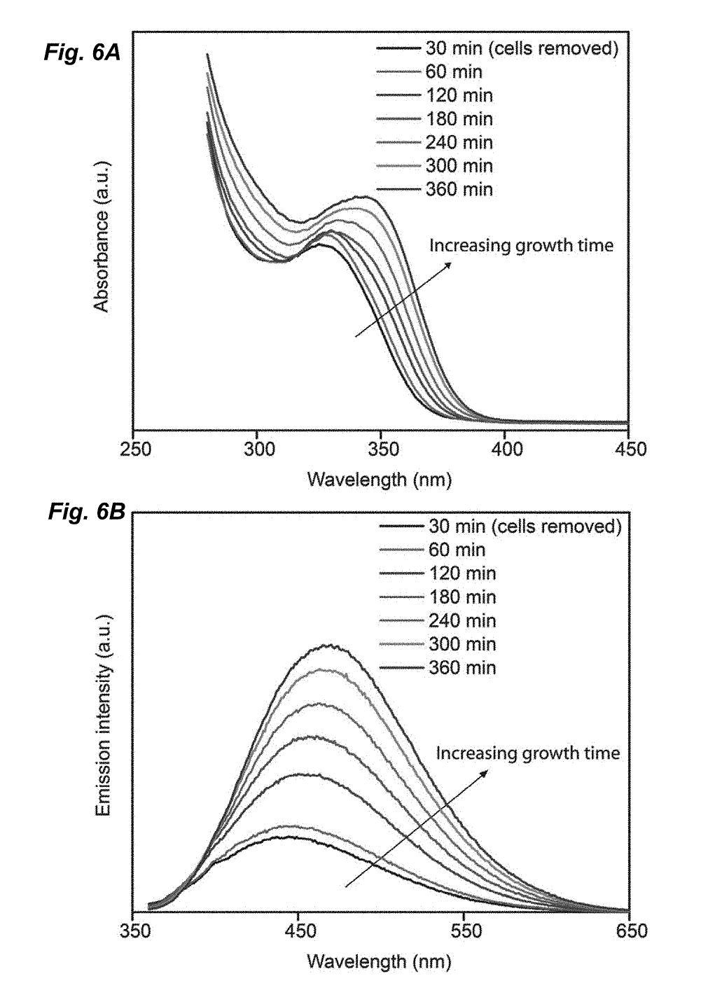

To further investigate the CdS growth mechanism, particle growth was investigated following the removal of the bacterial cells from culture via centrifugation (FIGS. 6A and B). After centrifugation, the optical density at 600 nm (OD600) of the supernatant, a measure of cell concentration, was <2% of the initial value, confirming removal of the cells. The CdS QDs in the free supernatant continued to grow in the absence of cells, although at a slower rate as characterized by a smaller red-shift in both adsorption and fluorescence peak maxima with increasing growth time. For example, after 360 minutes the absorbance and fluorescence maxima are at 343 and 469 nm, respectively, without cells and at 378 and 562 nm, respectively, with cells. This result both confirms the extracellular production of the QDs, and indicates that QD growth does not require the continuous presence of cells throughout the entire growth process although the presence of cells accelerates the rate of QD biosynthesis by continuously generating the extracellular components responsible for QD biosynthesis. Removal of the cells after an initial period reduces the rate of QD biosynthesis by reducing the concentration of extracellular components necessary for QD biosynthesis.

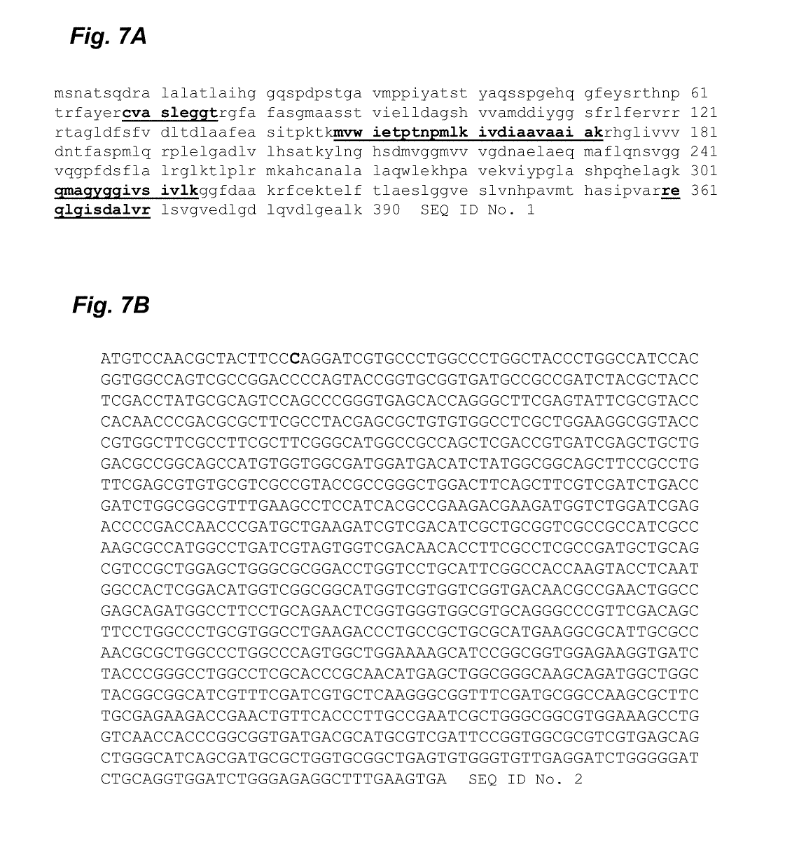

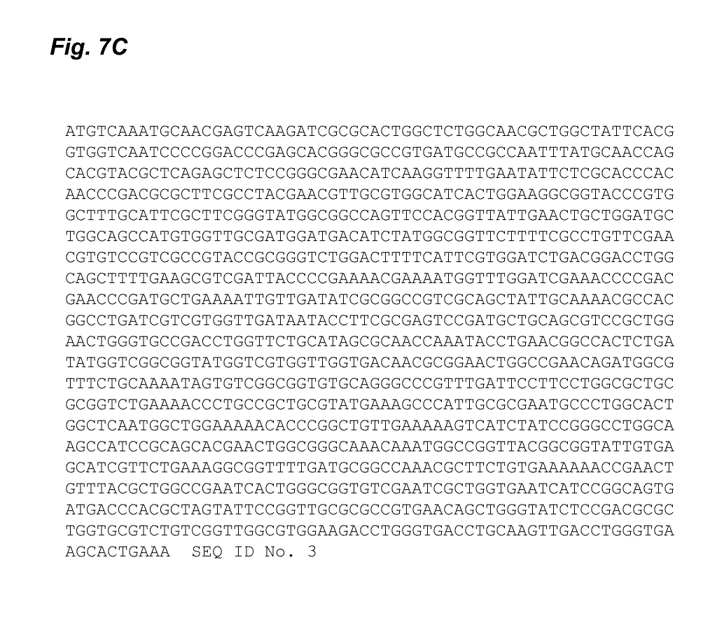

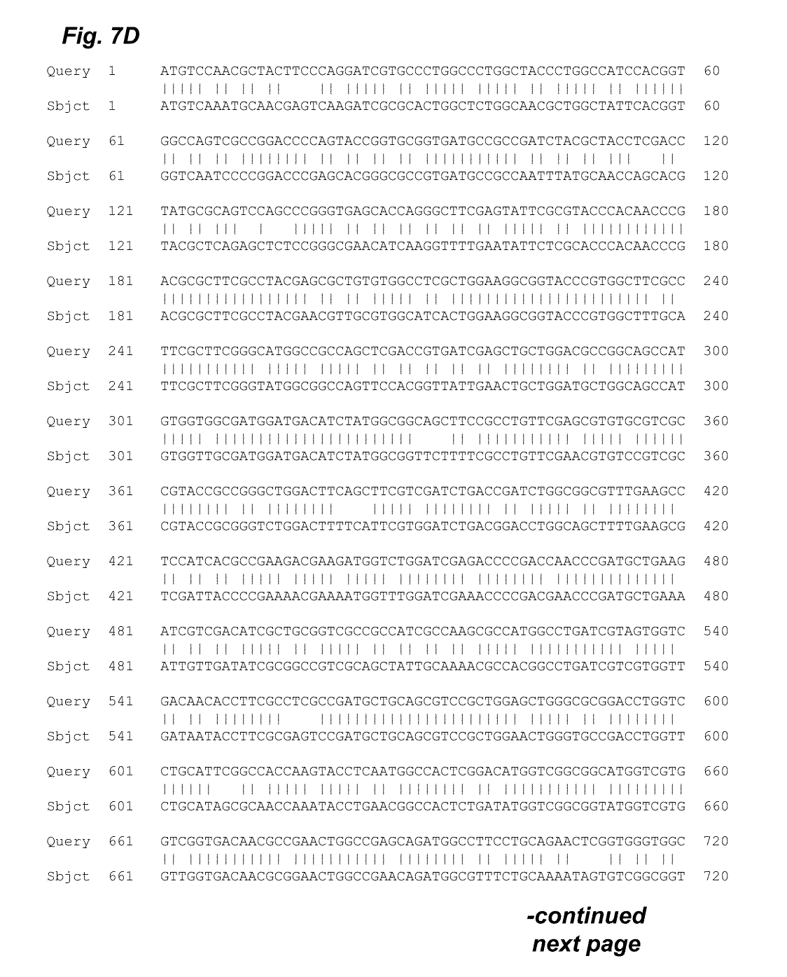

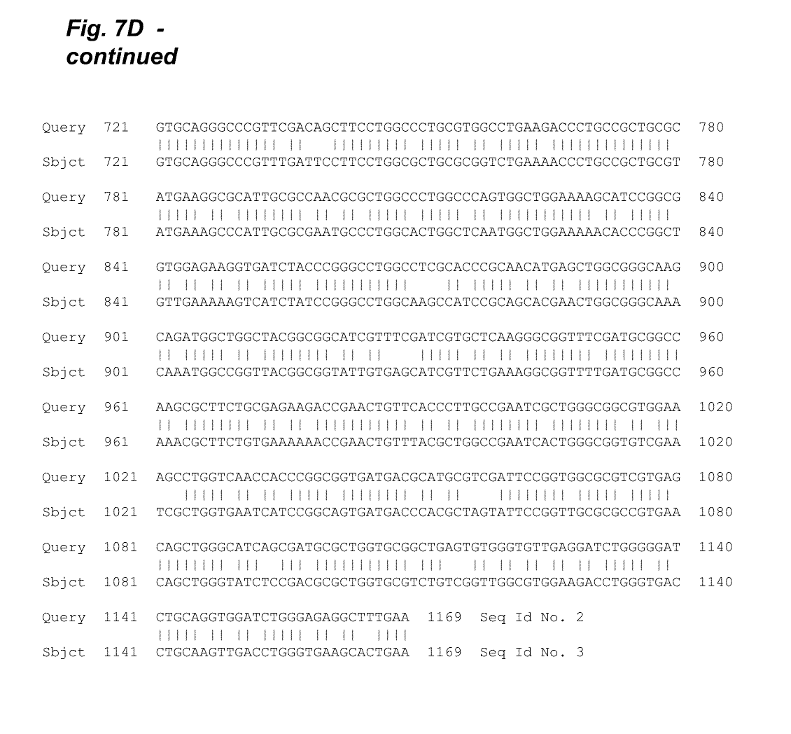

Interestingly, SDS-PAGE analysis of the supernatant did not show significant (>0.1-1.0 microgram detection limits) concentration of associated proteins or other biomacromolecules indicating that the QDs are free in solution and not entrained in extracellular matrix or cell remnants. Many of the prior reports on this topic describe intercellular nanocrystals production requiring cell lysis prior to purification. This leads to association of proteins and other intracellular biomacromolecules on the nanocrystal surfaces at higher (>0.1-1.0 microgram) concentrations. In order to determine if low-abundance proteins or other biomacromolecules are associated with the biosynthetic QDs produced by the method described in this embodiment, the QD containing supernatant was dialyzed against distilled water to reduce the free Cd salt and L-cysteine concentration, lyophilized and analyzed by electrospray ionization mass spectrometry (ESI-MS). This technique is significantly more sensitive than the SDS-PAGE and revealed several proteins associate with the QDs. Of particular note, a putative cystathione gamma-lyase (NCBI Accession Number WP_012509966) was identified from independent QD samples analysed by ESI-MS. The amino acid sequence of the identified molecule is shown in FIG. 7A.

Cystathione gamma (γ)-lyases are a class of enzymes that produce H₂S from L-cysteine, and prior work has shown that overexpression of a highly active cystathione gamma-lyase in E. coli confers resistance to otherwise toxic concentrations (0.1-0.4 mM) of aqueous cadmium chloride by precipitation of bulk CdS through generation of H₂S from 1 mM L-cysteine. As provided herein, a novel approach is exemplified for the reproducible biosynthesis of extracellular, water-soluble CdS QDs using an engineered strain of Stenotrophomonas maltophilia (SMCD1) that has been specifically evolved to control particle size. As such, one embodiment provides for room temperature and aqueous synthesis conditions enabling low-cost, green synthesis of such CdS QDs.

### EXAMPLE 2

### Generation of QD by Isolated Enzymes

In another embodiment, an engineered cystathionine γ-lyase (smCSE) is utilized to generate controlled CdS nanocrystal synthesis directly from aqueous solution using L-cysteine and cadmium acetate as reactants. The ability of smCSE to mineralize CdS and template nanocrystal formation provides a single enzyme route for engineered nanocrystal biomineralization.

The genomic sequence of S. maltophilia CSE (Smal_0489, Genscript, SEQ ID No. 7B) was codon optimized for expression in E. coli and was sub-cloned into pET28a (+) and transformed into BL21 E. coli cells as a host heterologous to the Stenotrophomonas geneus from which the gene was isolated. FIG. 7B shows the genomic sequence of the S. maltophilia CSE (Smal_0489), SEQ ID No. 2. FIG. 7C shows the nucleic acid sequence of S. maltophilia CSE as codon optimized for expression in E. coli. FIG. 7D provides an alignment of the genomic sequence and the codon optimized sequence. In this exemplified sequence the overall homology between the genomic sequence and the codon optimized sequence is 80% over 1169 nucleotides although the codon optimization could be performed to optimize a greater or fewer number of codons and be within the scope of the invention.

The host vector to which the gene was cloned includes a cloning site that places sequences encoding a His tag prior to a stop codon. Thus the histidine repeats on the COOH terminus of the amino acid sequence permit purification by metal affinity chromatography as described in Petty K J. Metal-Chelate Affinity Chromatography. Curr Protoc Protein Sci 4 (9.4) (2001) 1-16. Detection of H₂S product formation using the substrate L-cysteine was performed to determine Michaelis-Menten rate parameters was as described in Chiku T, et al. H₂S Biogenesis by Human Cystathionine Gamma-Lyase Leads to the Novel Sulfur Metabolites Lanthionine and Homolanthionine and Is Responsive to the Grade of Hyperhomocysteinemia. J Biol Chem 284(17) (2009) 11601-11612.

For biosynthesis of cadmium sulfide nanocrystals, purified smCSE (0.1 mg/mL) was incubated with 0.5 mM cadmium acetate and 4 mM L-cysteine in 100 mM Tris buffer (pH 7.5). CdS nanocrystal formation was directly observed in solution as a function of time by illumination using a UV lamp. Absorbance spectra for CdS nanocrystals were taken at regular time intervals on a Shimadzu UV-Vis 3300 spectrometer, and fluorescence emission spectra were measured on a PTI QuantaMaster fluorimeter with a 350 nm excitation wavelength using a 5 nm excitation slit width. To demonstrate the ability of CSE to control CdS nanocrystal growth and size independent of H₂S generation, Na₂S was added instead of L-cysteine to a final concentration of 4 mM in a solution containing 0.5 mM cadmium acetate and 0.1 mg/mL purified smCSE. Absorbance and fluorescence spectra as well as direct observation of solutions under UV light were used to characterize the optical properties of the CdS nanoparticles in solution. For X-Ray diffraction studies, it was found that incubation of smCSE with cadmium acetate and L-cysteine for 16-24 hours led to the formation of solutions of CdS particles displaying optical characteristics indicative of bulk as opposed to nanoscale material. This bulk CdS was collected by centrifugation and washed twice with absolute ethanol and dried under ambient conditions. Powder XRD spectra were taken of the resultant dried material on a Rigaku Miniflex II Diffractometer using Cu Kα (1.542 Å) radiation. Spectra were compared to standard XRD patterns for wurzite type CdS (PDF card no. 00-006-0314) and zinc blende type CdS (PDF card no. 00-010-0454) from the ICDD database.

For transmission electron microscopy (TEM) studies, biosynthetic CdS nanocrystals were grown for 3 hours, corresponding to an absorbance maximum at 350 nm, and subsequently dialyzed at 4° C. against a reservoir containing 0.5 mM L-cysteine for 24 h. The dialyzed CdS nanocrystals solutions were placed dropwise onto argon (Ar) plasma-treated carbon-coated copper TEM grids and the liquid evaporated under vacuum. HR-TEM and HAADF-STEM images were taken on a 200 kV aberration corrected JEOL ARM 200CF analytical electron microscope equipped with a Centurio XEDS system.

In order to determine whether the putative CSE that we identified from S. maltophilia (smCSE) was capable of H₂S generation from L-cysteine, smCSE was heterologously overexpressed and purified from E. coli and the intrinsic kinetics of L-cysteine turnover to H₂S measured. Expression and purification using immobilized metal affinity chromatography yielded a single protein of 42 kDa, consistent with the expected size of smCSE (NCBI reference number WP_012509966.1). The purified smCSE also shows a strong absorbance peak at 430 nm, which is indicative of a covalently-bound pyridoxal 5′ phosphate (PLP) co-factor. PLP is a known obligate co-factor required for CSE catalysis. The Michaelis-Menten rate parameters for L-cysteine turnover measured for smCSE using L-cysteine as a substrate was determined to be consistent with reported values for other well-characterized CSEs (data not shown).

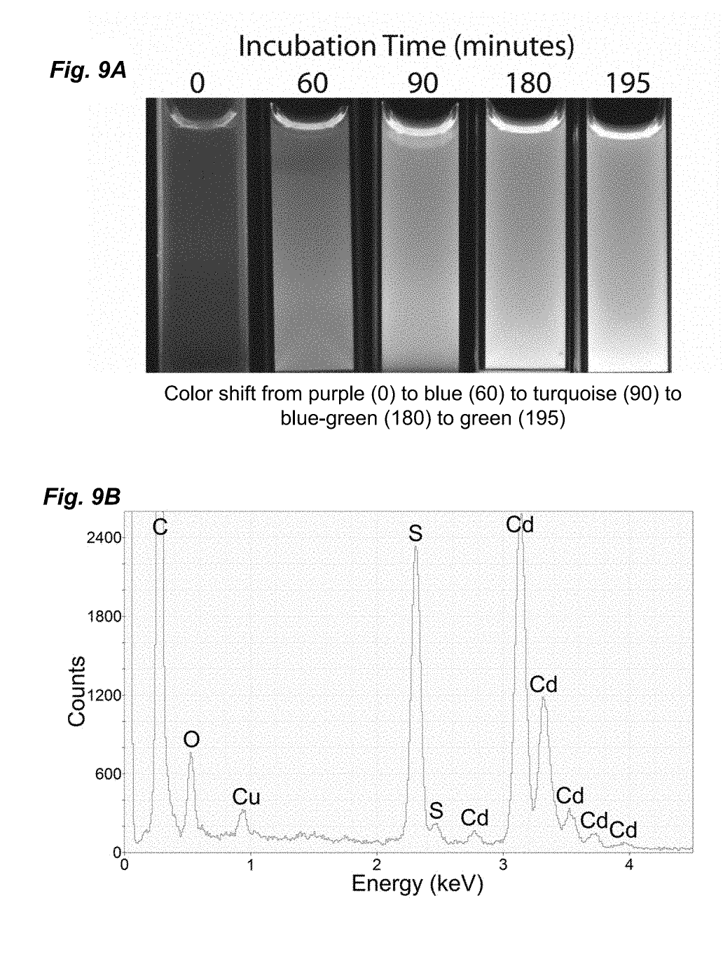

Having established the ability of smCSE to generate reactive sulfur species, it was hypothesized that the addition of a cadmium salt to the solution would yield CdS. In order to test this hypothesis, L-cysteine (4 mM) and cadmium acetate (0.5 mM) were added to an aqueous solution of purified smCSE (0.1 mg/mL in Tris buffer, pH 7.5), and the solution monitored using UV-visible and fluorescence spectroscopy as a function of time at ambient temperature and pressure (FIGS. 8A and B). After 90 minutes, a distinct absorbance peak was observed with an absorbance maximum at 333 nm (FIG. 8A). A corresponding fluorescence emission peak at 464 nm was also observed (FIG. 8B). The maxima of both absorbance and fluorescence progressively shift to longer wavelengths over the course of 195 minutes (FIGS. 8A and B) with visible photoluminescence observed in solution for each time point measured when illuminated under UV light (FIG. 9A). It should be noted that the presence of smCSE, cadmium acetate and L-cysteine were all required; omission of any one of these components produced solutions that displayed no absorbance or fluorescence peaks and no observable photoluminescence when illuminated under UV light. Placing the reactant mixture on ice essentially arrests smCSE activity allowing a specific population of particles with a given set of photoluminescent properties to be collected.

Enzymatically synthesized CdS QDs were determined to be monodisperse and crystalline. Crystallites were harvested from solution after 180 minutes of growth (absorbance maximum at 350 nm) and analyzed via scanning transmission electron microscopy (STEM). FIG. 10A shows the existence of discrete, but irregularly shaped and overlapping nanocrystals, between 2-4 nm in diameter. Elemental analysis from this region using X-ray energy-dispersive spectroscopy (XEDS) (FIG. 9B) reveals strong Cd and S signals. The presence of strong Cu and C peaks are artefacts arising from the copper mesh TEM grid coated with a carbon film. High-resolution high-angle annular dark field (HAADF) imaging of individual and isolated particles confirms the size of the nanocrystals and the presence of both wurtzite (FIG. 10B) and zinc-blende structured (FIG. 10C) nanocrystals. The lattice parameters and inter-planar angles of these crystals are consistent with those for CdS. These lattice parameters and both crystalline phases have previously been observed for chemically- and biologically-synthesized CdS nanocrystals. Powder X-ray diffraction measurements on material harvested after 12 hours growth was also consistent with the presence of both structures of CdS.

The irregular shape of the biomineralized nanocrystals prevents a precise comparison with reported literature correlations for the optical properties as a function of particle diameter. However, for all reported growth times the absorbance maximum (FIG. 8A) remains well below the absorbance maximum corresponding to the band gap of bulk CdS (2.5 eV, 495 nm) and the observed biomineralized CdS nanocrystals size is well within the quantum confined size range for CdS. See Alivisatos A Semiconductor Clusters, Nanocrystals, and Quantum Dots. Science 271(5251) (1996) 933-937. The observed red-shift in optical properties with growth time (FIGS. 8A and B) indicates CdS nanocrystal growth as a function of time within this quantum-confined size range. As demonstrated herein, adsorption peak maxima of 324, 334 and 344 nm correspond to biomineralized nanocrystallite sizes of 2.75±0.68, 3.04±0.75, and 3.36±0.95 nm, respectively. Additionally, smCSE produced CdS nanocrystals show similar quantum yield (1.8%) as compared to synthesized CdS nanocrystals from S. maltophilia (2%) as described herein.

CdS QD growth was shown to be dependent on both H₂S production and available capping agents. While L-cysteine is a substrate for smCSE to generate H₂S, it can also act as an aqueous phase capping agent for CdS. To confirm this dual role of L-cysteine as sulfur source and capping agent, CdS biomineralization was studied as a function of L-cysteine concentration. At a concentration of 4 mM L-cysteine, found to be the practical lower limit for nanocrystal synthesis at 0.1 mg/mL smCSE and 0.5 mM cadmium acetate, the observed optical absorbance maximum increases with time, reaching 370 nm after 4 h of growth (FIG. 11A); longer growth times beyond 4 h led to formation of bulk CdS with an absorbance maximum consistent with bulk CdS. Increasing L-cysteine concentration to 10 mM and 20 mM red-shifts the absorbance maximum and corresponding photoluminescence, during growth (FIGS. 11A and 11B). This is indicative of an increase in CdS growth rate with increasing L-cysteine concentration for nanocrystals within the quantum confined size range and is particularly noticeable for the 20 mM L-cysteine data. Interestingly all of the growth curves reach similar maxima after four hours, 370, 375, and 380 nm at 4, 10 and 20 mM L-cysteine, respectively. This is commensurate with the loss of optical clarity of the solutions at longer growth times, indicating a maximum in the solubility of the presumably L-cysteine capped nanocrystals; images of solutions illuminated using UV light at each L-cysteine concentration and growth time are given in FIG. 11B to demonstrate optical clarity at each solution condition.

To further demonstrate that thiol-mediated capping is important for stabilizing the nanocrystals, glutathione was introduced into the enzymatic synthesis mixture. Glutathione is a capping agent derived from L-cysteine and L-glutamine that has been shown previously to stabilize water-soluble CdS nanocrystals. However, unlike L-cysteine, glutathione is not a substrate for CSEs, and therefore acts solely as a capping agent. No nanocrystal formation was observed in the absence of L-cysteine. This clearly demonstrates that L-cysteine is the sulfur source for the nanocrystal growth through the enzymatic generation of H₂S.

In the presence of 4 mM L-cysteine, 1 mM and 4 mM glutathione addition yielded growth curves and absorbance maxima at four hours, (375 and 385 nm for 1 mM and 4 mM glutathione, respectively), similar to those with elevated L-cysteine concentration although the solutions maintain optical clarity after five hours of growth (FIGS. 11c and 11d). This is indicative of increased stabilization of the aqueous nanocrystal solution with the glutathione capping agent versus L-cysteine capping agent. While the 4 mM glutathione growth curve shows some indication of a decreased growth rate, this phenomenon is clearly observed upon increasing the glutathione concentration to 10 mM. When compared with lower glutathione concentration, the 10 mM glutathione growth curve shows a blue shift in absorbance maximum at all growth times and yields an optically clear solution with absorbance maximum at 400 nm after twenty four hours growth (FIGS. 11c and 11d).

In order to further demonstrate the role of glutathione as a capping agent, the absorbance maximum and L-cysteine concentration were measured as a function of time in the presence and absence of glutathione. While L-cysteine degradation is not influenced by the presence of glutathione, the absorbance maximum occurs at lower wavelength, indicating a smaller average particle size, for the glutathione containing sample. This is consistent with capping role envisioned for glutathione as an increased capping agent concentration will lead to smaller average particle size. Where the L-cysteine concentration dependent stabilization and growth of CdS nanocrystals is due to its role as both a capping agent and substrate for smCSE, addition of a thiolated capping agent glutathione that is not a substrate for smCSE stabilizes the nanocrystal solution and shifts the crystal growth curve to a lower average size at a given time.

smCSE was shown to regulate CdS nanocrystal growth and size independent of H₂S generation. To ascertain the role of smCSE in directing the structure and size of the CdS nanocrystals, L-cysteine was replaced with Na₂S to act as the sulfur source. Na₂S is not a substrate for smCSE, such that H₂S will not be produced by smCSE under these conditions, but Na₂S is a sulfur source in the aqueous chemical synthesis of CdS nanocrystals in the presence of thiolated ligand capping agents. Addition of Na₂S (4 mM) to an aqueous solution of cadmium acetate (0.5 mM) at room temperature lead to nearly instantaneous formation of large aggregates of bulk CdS, with no distinct absorbance maximum (FIG. 12A or photoluminescence (FIG. 12B). This is due to the lack of a capping agent to control nanocrystal growth. However, when smCSE (0.1 mg/mL) is added to the solution, a distinct absorbance maximum rapidly emerges at 360 nm (FIG. 12A), indicating the formation of CdS nanocrystals. This clearly indicates the role of smCSE in templating and controlling the growth of CdS nanocrystals.

Absorbance and photoluminescence characteristics indicative of nanocrystal formation were also observed if known capping agents L-cysteine or glutathione were present (FIGS. 12A and 12B). The absorption maxima for the glutathione and L-cysteine capping ligands are sharper, which likely reflects their higher affinity for the particle surface and their smaller size, both of which lead to higher coverage on the particle surface during growth. Thus, in all 3 cases, smCSE, L-cysteine and glutathione act as structure directing agents to form CdS nanocrystals, rather than bulk CdS, from Na₂S and cadmium acetate. This clearly demonstrates that that smCSE has the intrinsic ability to regulate CdS nanocrystal growth independently from, and in addition to, its role in reactive H₂S generation. FIG. 12C is a schematic of proposed CdS quantum dot synthesis by smCSE: first, smCSE associates with cadmium acetate and L-cysteine present in solution, smCSE then degrades L-cysteine to produce H₂S. The H₂S produced by smCSE nucleates CdS nanoparticles and CdS nanoparticles continue to grow upon the continuous generation of H₂S (FIG. 12C).

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A method of biosynthesizing a nanoparticle quantum dot material comprising:
providing a bacterial organism that is tolerant to a selected metal salt;
placing the bacterial organism in an aqueous environment comprising the selected metal salt for a time sufficient for the bacterial organism to utilize the metal salt to assemble semiconductor nanoparticles;
removing the bacterial organism to provide a cell-free solution;
allowing the semiconductor nanoparticles to continue to grow in the cell-free solution until a desired semiconductor quantum dot population is obtained, and
harvesting the desired semiconductor quantum dot population.

2. The method of claim 1, wherein the semiconductor nanoparticles have an average particle size of between about 1 nm to about 10 nm.

3. The method of claim 2, wherein the bacterial organism is selected from a class Gammaproteobacteria and an order Xanthomonadales.

4. The method of claim 3, wherein the bacterial organism is selected from a genus Stenotrophomonas.

5. The method of claim 4, wherein bacterial organism is a species selected from: S. acidaminiphila, S. dokdonensis, S. koreensis, S. maltophilia, S. nitritireducens, and S. rhizophila.

6. The method of claim 1, wherein the metal salt is selected from I-VI, II-VI, IV-VI and III-V semiconductor metals.

7. The method of claim 6, wherein the metal salt comprises cadmium, and wherein the bacterial organism is tolerant to cadmium concentrations above 1 mM.

8. A semiconductor quantum dot produced by the method of claim 1.

9. A method of generating an enzyme that biosynthesizes nanoparticle quantum dot materials comprising:
iteratively culturing and selecting a bacterial organism that is tolerant to growth in the presence of a metal salt that is selected from I VI, II-VI, IV-VI and III-V semiconductor metals and generates controlled particle size quantum dots by culture in the presence of the metal salt;
isolating and cloning a gene encoding a cystathione gamma (γ)-lyase from the tolerant bacterial organism;
expressing the gene from a heterologous host organism;
isolating the cystathione gamma (γ)-lyase produced by the heterologous host organism.

10. The method of claim 9, wherein the gene is codon optimized for expression by the heterologous host organism.

11. The method of claim 9 wherein the bacterial organism is selected from a class Gammaproteobacteria and an order Xanthomonadales.

12. The method of claim 9, wherein the bacterial organism is selected from a genus Stenotrophomonas.

13. A method of biosynthesizing a nanoparticle quantum dot material comprising incubating a cystathione gamma (γ)-lyase in an aqueous solution comprising L-cysteine and cadmium acetate for a sufficient time to generate a desired quantum dot material and isolating the quantum dot material.

14. The method of claim 13, wherein the cystathione gamma (γ)-lyase is a recombinantly produced enzyme isolated from a Stenotrophomonas bacteria that was selected for tolerance to growth in the presence of cadmium.

15. The method of claim 14, wherein the bacteria is a Stenotrophomonas maltophilia bacteria.

16. A biosynthetic nanoparticle quantum dot material, wherein the quantum dot material is enzymatically synthesized by an isolated recombinant cystathione gamma (γ)-lyase in an aqueous solution comprising L-cysteine and a metal salt.

17. The biosynthetic nanoparticle material of claim 15, wherein the metal salt comprises a metal selected from the group consisting of Cd, Ce, Cu, Fe, Hg, In, Ga and Zn.

18. The biosynthetic nanoparticle material of claim 16, wherein the metal salt comprises a Cd metal.

19. The biosynthetic nanoparticle material of claim 18, wherein the metal salt is a cadmium acetate.

20. A codon optimized gene encoding a recombinant cystathione gamma (γ)-lyase wherein the gene is isolated from a Stenotrophomonas bacteria and is codon optimized for expression in a heterologous host.

21. The codon optimized gene of claim 20, wherein the heterologous host is E. coli.

22. An isolated nucleic acid comprising a yeast or E. coli codon optimized nucleotide sequence that encodes a cystathione gamma (γ)-lyase isolated from a Stenotrophomonas bacteria that was selected for tolerance to growth in the presence of cadmium.
